(19) United States
(12) Patent Application Publication
(10) Pub. No.: US 2025/0377273 A1
(43) Pub. Date: Dec. 11, 2025

(54) METHOD FOR DIAMONDOID ISOLATION FROM PETROLEUM AND ROCKS

(71) Applicant: Saudi Arabian Oil Company, Dhahran (SA)

(72) Inventor: Ibrahim Z. Atwah, Dhahran (SA)

(21) Appl. No.: 18/740,093

(22) Filed: Jun. 11, 2024

(51) Int. Cl. G01N 1/40; G01N 30/02; G01N 30/12

(52) U.S. Cl. CPC G01N 1/4055; G01N 30/12; G01N 2001/4061; G01N 2030/025; G01N 2030/027

(57) ABSTRACT

The technology relates to the isolation and purification of diamondoids from a hydrocarbon sample. The hydrocarbon sample can include rock samples or petroleum fluid samples. The technology involves preparation, isolation, and analysis. The preparation involves the extraction of organic compounds from the hydrocarbon sample, resulting in an extract. From the extract, a combination of liquid chromatography and gas chromatography is used to isolate the diamondoids. The isolated diamondoids are further purified using molecular sieves. The isolated diamondoids are analyzed using a gas chromatography-mass spectrometry to identify the type of diamondoid.

### TECHNICAL FIELD

This disclosure relates to a method of diamondoid isolation from petroleum samples and rocks.

### BACKGROUND

Diamondoids are unique molecules with a cage-like structure that have a similar structure to the diamond lattice but are much smaller. They are found naturally in petroleum and certain geological formations rich in organic material. Their structure gives them a level of stability and strength comparable to materials like graphite and carbon nanotubes. Their application and utilization spans across many industries due to unique physio-chemical properties, one of which is ultra thermal stability.

In industries related to materials science and petroleum, diamondoids can be added to materials to make them stronger and more durable. Given the natural occurrence of diamondoids in petroleum fluids and rocks, their direct extraction from these sources provides a cost effective approach to supply diamondoid raw material, as compared to chemical synthesis which would require raw material, chemical, and energy to synthesize in the laboratory.

However, due to their low abundance in petroleum and rock samples, isolating diamondoids can be challenging. Isolation of diamondoids require large sample mass or certain thermal maturity to achieve desired isolation results.

### SUMMARY

The present disclosure provides a method for the extraction and purification of diamondoid compounds. In some implementations of the method, a rock sample or a petroleum fluid sample (hydrocarbon sample) that includes several diamondoids is obtained from a well rig or refinery. In some implementations, the hydrocarbon sample is subjected to programmed temperature and pressure cycles. In some implementations, an extraction process removes several organic compounds from the rock sample using solvent extraction by a first solvent, resulting in an extract. In some implementations, the extract includes several diamondoids. In some implementations, a saturated hydrocarbon fraction is separated from the extract using liquid chromatography (LC) that uses a second solvent. In some implementations, several diamondoids are isolated from the saturated hydrocarbon fraction using a column packed with a high sorbent phase and using LC. In some implementations, the diamondoids are released from the high sorbent phase by a sequence of steps that involve gas chromatography (GC), LC, and molecular sieves. In some implementations, the diamondoids are analyzed by a gas chromatography-mass spectrometry (GC-MS) to identify the type of diamondoid isolated.

Implementations here provide a diamondoid isolation device that includes an extraction vessel, where the rock sample of the hydrocarbon sample undergoes a solvent extract process, resulting in an extract. In some implementations, a first column receives the extract, from which a saturated hydrocarbon fraction is removed using LC. In some implementations, an evaporation column receives the saturated hydrocarbon fraction from the first column, and in the evaporation column a mixture of hydrocarbons fraction is formed. In some implementations, a second column includes a high sorbent stationary phase, which is configured to receive the mixture of hydrocarbons fraction. In some implementations, several diamondoids are isolated from the mixture of hydrocarbons fraction using a combination of LC and GC. In some implementations, a third column that includes molecular sieves is further used to purify the isolated diamondoids.

Implementations provided here relate to a method of diamondoid isolation from a rock sample or a petroleum sample (hydrocarbon sample), obtained from an oil well rig or refinery. In some implementations, diamondoids are present in the hydrocarbon sample and are isolated by a combination of LC, GC, and further purified using molecular sieves. In some implementations, the obtained diamondoids are analyzed by GC-MS to identify the type of diamondoid obtained.

### BRIEF DESCRIPTION OF DRAWINGS

FIG. 1 is a drawing of a three-step process for a diamondoid isolation and purification method.

FIG. 2 is a schematic representation of an exemplary diamondoid isolation process.

FIGS. 3A-3F represent a total ion chromatogram (TIC) of crude oil and rock extracts before and after diamondoid isolation. FIGS. 3A-3C show diamondoids isolated from crude oil, where FIG. 3A is a chromatogram showing crude oil before isolation; FIG. 3B is a chromatogram showing isolated diamondoids from crude oil; and FIG. 3C is the mass spectrum of adamantane. FIGS. 3D-3F show diamondoids isolated from rock extracts, where FIG. 3D is a chromatogram showing rock extracts before isolation; FIG. 3E is a chromatogram showing isolated diamondoids from rock extracts; and FIG. 3F is the mass spectrum of diamantane.

FIG. 4 is a process flow diagram representing the exemplary diamondoid isolation process.

### DETAILED DESCRIPTION

Implementations of the present disclosure provide a method for the extraction and purification of diamondoid compounds. In some embodiments, the steps include preparation, isolation, and analysis of high purity diamondoids. The preparation step can include obtaining samples from oil rigs and refineries for diamondoid extraction. The preparation step can also involve a solvent extraction process for the rock samples resulting in a complex matrix of crude oil or rock extracts, also termed as extract. Petroleum fluid samples do not need an extraction process and can undergo liquid chromatography (LC) directly. LC can be used to separate the saturated hydrocarbon fraction from the extract and the petroleum fluid samples. In some embodiments, the solvent used in the LC is separated from the saturated hydrocarbon and further concentrated resulting in a mixture of normal alkanes (n-alkanes) and isoalkanes, cycloalkanes, and a plurality of diamondoid compounds. In some embodiments, LC includes the removal of asphaltenes, NSO polar compounds, and aromatic compounds. As used herein, “NSO polar compounds” refer to nitrogen, sulfur, and oxygen-containing compounds in crude oil that contribute to its polarity, including nitrogen heterocycles, sulfur heterocycles, and oxygen-containing compounds.

In implementations disclosed herein, the saturated hydrocarbon fraction undergoes an isolation step. The isolation step includes a combination of LC and gas chromatography (GC), molecular sieving, and the recovery of sieved compounds. In some implementations, after the preparation step, a selective evaporation process is used to remove the lower boiling compounds from the saturated hydrocarbon fraction. An aspect described here includes evaporation under reduced pressure to selectively remove solvents, which typically have a lower boiling point under vacuum than the plurality of diamondoids. Subsequent to selective evaporation, the saturated hydrocarbon fraction undergoes LC and GC. In an aspect described here, GC is conducted under a specific temperature regime and constant carrier gas flow.

In implementations described herein, the isolation step further includes molecular sieving to purify the plurality of diamondoids. In an aspect described here, the molecular sieves include zeolites. The zeolite pores selectively capture the plurality of diamondoid structures, allowing other hydrocarbons to be vented. Following the molecular sieving process, a solvent mixture is injected for further molecular sieving. This results in solvent extracts which are subjected to a high-frequency sonication and centrifugation to recover the plurality of diamondoids. The obtained plurality of diamondoids is of high purity. In some embodiments, the plurality of diamondoids is analyzed by gas chromatography mass spectrometry (GC-MS) to confirm the purity.

Implementations described herein, provide a system to isolate a plurality of diamondoids from rock samples or petroleum samples. In some embodiments, the plurality of diamondoids includes adamantane, diamantane, triamantane, and their alkylated variants. In some embodiments, the diamondoid isolation system includes an extraction vessel, a first column for LC, an evaporation column, a second column that includes a high-sorbent phase for a combination of LC and GC, and a third column that includes molecular sieves.

Diamondoids are small carbon cage-like structures which resemble the diamond lattice. They have carbon frameworks that can be superimposed on a diamond lattice. The smallest carbon cage is adamantane (C₁₀H₁₆), which includes one cage-shaped subunit. Diamondoids also include one or more cages, such as diamantane, triamantane, tertramantane, and higher polymantanes. The diamondoid compounds are nanometer in size. Higher diamondoids are in the micron sizes. For example, diamantane has two face-fused cages and triamantane has three face-fused cages.

The diamond faced fused cage structure provides stability, strength, and rigidity. However, synthesizing them in a laboratory is typically very difficult due to rigid structure and generally results in low yields. Additionally, potential intermediates and complex reaction kinetics makes the synthesis very challenging. However, none of the synthesis process offers a way to obtain a plurality of diamondoids ranging from adamantane to higher polymantanes from a versatile sample selection, such as disclosed herein. The method described herein allows for the isolation and purification of a plurality of diamondoids from sources such as petroleum fluids, crude oil, rock samples, and kerogen.

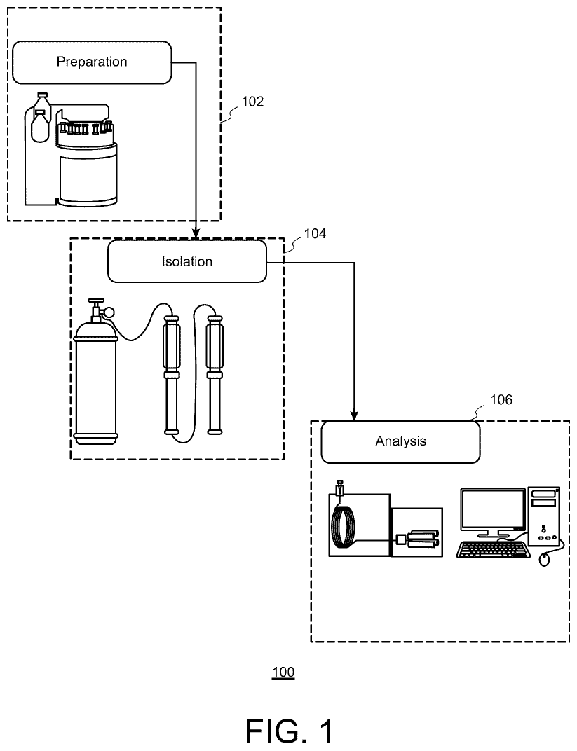

FIG. 1 is a drawing of a three-step process for a diamondoid isolation and purification method 100. The three steps for the diamondoid isolation include preparation, isolation and purification, and analysis. At block 102, the preparation of the sample involves obtaining a rock sample or a petroleum fluid sample. An aspect includes rock or petroleum fluid sample collection from various sources such as well rigs, encompassing rock core samples, drilling cuttings, soil samples, down-hole sampled petroleum fluids, or produced crude oil from rigs or refineries crude oil, or kerogen from well rigs, drill cuttings, soil samples, downhole sampled petroleum fluids, or produced crude oil from rigs or refineries.

For rock samples, preparation involves solvent extraction to liberate organic compounds from the rock matrix, including a plurality of diamondoids. This process entails powdering the rock, then subjecting the powdered rock to programmed temperature and pressure cycles in an extraction vessel using organic solvents. The organic solvents can include, but are not limited to, hexane, pentane, dichloromethane, isooctane, carbon disulfide, methanol, ethanol, and chloroform. This process results in a rock extract. Solvent choice and ratios vary based on the rock's mineralogy to maximize organic compound yield, including diamondoids. Post-extraction, solvents are reduced using wet chemistry techniques such as rotary evaporation. In some implementations, petroleum fluid samples can undergo a solvent extraction process. The preparation step can involve a tailored solvent extraction technique for both rock and petroleum fluid samples.

To prevent the loss of lower diamondoids, like adamantane and diamantane during the evaporation step, an optimized rotary evaporation system is utilized. This system operates under reduced pressure to selectively remove solvents that typically have a lower boiling point under vacuum than the lower diamondoids, like adamantane and diamantane. This method takes advantage of the distinct physical properties between the solvent and diamondoids, ensuring efficient separation without loss of target compounds. Furthermore, the evaporated solvents are condensed and subsequently analyzed using gas chromatography-mass spectrometry (GC-MS) to verify the absence of diamondoids. This evaporation procedure ensures that lower diamondoids are not lost in the preparation process.

Rock extracts, crude oils, and petroleum fluids undergo a subsequent preparation sequence involving liquid chromatography (LC) to isolate saturated hydrocarbons. This step employs chromatography columns packed with silica gel (or its derivatives) as the stationary phase. The sample is introduced atop the column and eluted with non-polar solvents. The non-polar solvents can include pentane, hexane, dichloromethane, carbon disulfide, octane, or isooctane. The non-polar solvent separates the saturated hydrocarbons from the complex matrix of crude oil, petroleum fluids, or rock extracts. The non-polar solvent is then removed from the saturated hydrocarbon fraction to concentrate the sample. The saturated hydrocarbon fraction contains a mix of normal alkanes (n-alkanes) and isoalkanes, cycloalkanes, and diamondoid compounds. The preparation stage is crucial for optimizing rock extraction parameters to enhance diamondoid recovery and purifying the complex matrix of rock extracts and petroleum fluids. This liquid chromatography preparation step removes fractions like asphaltenes, NSO polar compounds, resins, and aromatic compounds, thus preparing the saturated hydrocarbon fraction for the subsequent isolation step.

At block 104, the isolation process involves three primary mechanisms: gas and liquid chromatography, molecular sieving, and recovery of the sieved compounds. The hydrocarbon saturated fraction obtained from the preparation step in block 102 is introduced into an evaporation column. The evaporation column is equipped with a heating element. This set up facilitates selective evaporation of lower boiling compounds via a temperature controlled program, along with a regulated flow of inert carrier gas such as nitrogen, helium, or argon.

After the selective evaporation process, the saturated hydrocarbon fraction is moved to a second column packed with a high-sorbent stationary phase. The stationary phase is composed of a mixture including but not limited to silica gel, nanotube materials, activated carbon, and hyper-crosslinked polymeric resins, designed to enhance diamondoid retention. Once the saturated hydrocarbon fraction is trapped in the second column, valves are adjusted to facilitate gas chromatography (GC) under a specific temperature regime and constant carrier gas flow. GC is used to achieve precise isolation of diamondoid compounds by leveraging the temperature-programmed separation. GC can operate at higher temperatures that are required for an effective isolation process. This process aides in the release of the sorbed compounds, including diamondoids, into a third column filled with activated molecular sieving materials. The molecular sieves include variously proportioned zeolites. The zeolite pores selectively capture diamondoid structures, allowing other hydrocarbons in the saturated hydrocarbon fraction to be vented.

Following the temperature controlled program of the GC, the system switches to a liquid chromatography phase. A solvent mixture is pumped from a reservoir via a pump through the second column into the third column for further diamondoid sieving. Post this liquid chromatography phase, the zeolite materials are immersed in the solvent mixture containing the extracts and subjected to high-frequency sonication. This facilitates the liberation of the trapped diamondoids. The solvent mixture is then centrifuged to separate the zeolite powder from the diamondoid extract, followed by rotary evaporation to concentrate the diamondoids. The rotary evaporation operates under reduced pressure to selectively remove solvents, which typically have a lower boiling point under vacuum than the lower diamondoids. This method takes advantage of the distinct physical properties between the solvent and diamondoids, ensuring efficient separation without the loss of target compounds.

This isolation process yields high purity diamondoids. A plurality of diamondoids including adamantane, diamantane, triamantane, and their alkylated variants are isolated by this process.

At block 106, the analysis process includes the use of a GC-MS to analyze the solvent mixture from the previous step. The solvent mixture after the evaporation process is condensed and analyzed by the GC-MS to verify the absence of diamondoid compounds. Further the diamondoid concentrate is analyzed to identify the molecular structure of the obtained diamondoids from the rock extracts and petroleum samples. The obtained plurality of diamondoid compounds can be subsequently separated into distinct compounds using high performance liquid chromatography (HPLC) method.

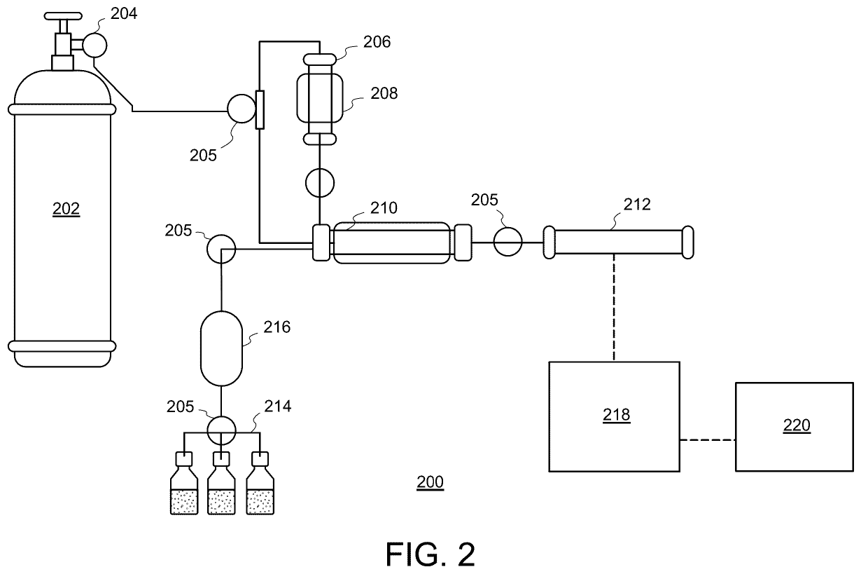

FIG. 2 is a schematic representation of the diamondoid isolation process 200.

The isolation device includes a gas cylinder 202 that is used to flow the carrier gas such as an inert gas. In some implementations, the carrier gas includes nitrogen, helium, or argon. A gas flow regulator 204 is used to adjust the flow of the carrier gas. A valve 205 is used to direct the flow of carrier gas either into the evaporation column 206 or the second column 210.

A saturated hydrocarbon fraction is prepared in the preparation step by extracting it from a rock sample, a crude oil sample, or a petroleum fluid sample. The saturated hydrocarbon fraction includes a mixture of alkanes and diamondoid compounds. The saturated hydrocarbon fraction, post the preparation step is introduced into the evaporation column 206. The evaporation column 206 is equipped with a heating element 208. This set up facilitates selective evaporation such that the lower boiling compounds from the saturated hydrocarbon fraction are vaporized via a temperature-controlled program, alongside a regulated flow inert carrier gas.

Subsequent to evaporation, the saturated hydrocarbon fraction traverses to a second column 210. The second column 210 is packed with a high sorbent stationary phase. The stationary phase includes a mixture of silica gel, nanotube materials, activated carbon, and hyper crosslinked polymeric resins such that the stationary phase can enhance diamondoid retention. The second column has valves 205 that can be adjusted to operate by LC method or GC method. GC is conducted under a specific temperature regime and constant carrier gas flow. The carrier gas flows from the gas cylinder 202 via the valve 205 into the second column 210.

Liquid chromatography and gas chromatography are used to separate molecules in complex mixtures. LC operates in the range of 20-30° C. However, GC can operate at higher temperatures and is used for targeted vaporization and isolation of specific compounds. Therefore, a combination of LC and GC is an effective way for the isolation of a plurality of diamondoids from adamantane to higher polymantanes.

Once the saturated hydrocarbon fraction is trapped in the second column 210, valve 205 is adjusted to facilitate GC in the second column 210. GC operates under a programmed temperature regime. It has a constant carrier gas flow. GC helps to release the retained compounds from the stationary phase of the second column 210, which include a mixture of a plurality of diamondoids and saturated hydrocarbons, into the third column 212. The third column 212 is packed with activated molecular sieves. Molecular sieves have uniform pore sizes and selectively absorb molecules based on molecular size. Typically, smaller molecular species have a higher diffusion rate. In implementations here, the third column 212 includes variously proportioned zeolites. The zeolite pores selectively capture diamondoid structures, allowing other hydrocarbons to be vented out.

Following the programmed temperature completion in the second column 210, the system switches to a LC phase. A solvent mixture in a dispenser 214 is pumped via a liquid pump 216 through the second column 210 into the third column 212 for further diamondoid isolation by the zeolites. Post chromatography, the zeolite materials are immersed in the solvent extracts and subjected to high frequency sonication in a sonication unit 218, facilitating the release of the trapped diamondoids. The mixture is then centrifuged in a centrifugation unit 220 to separate the zeolite powder from the diamondoid extract, followed by rotary evaporation to concentrate the diamondoids. This intricate process yields high purity diamondoids, including adamantane, diamantane, triamantane, and their alkylated variants. These diamondoids can subsequently be separated into distinct compounds using conventional HPLC methods.

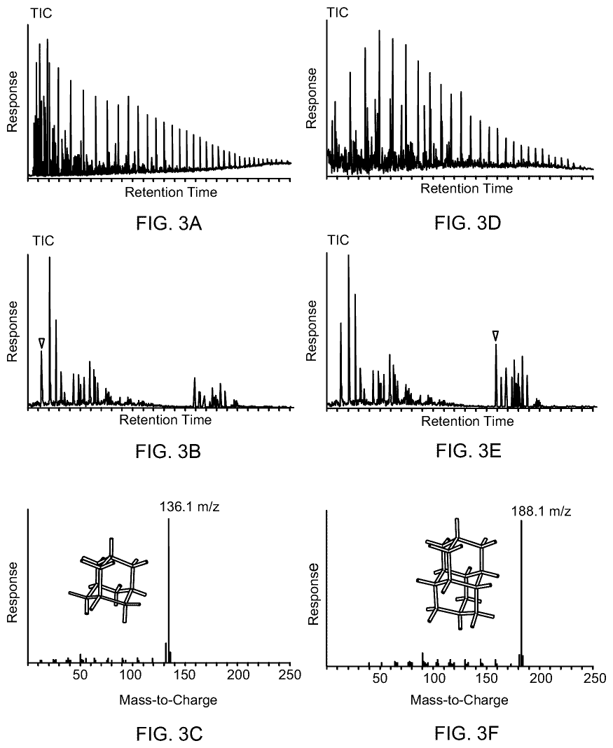

FIGS. 3A-3F represent a total ion chromatogram (TIC) of crude oil and rock extracts before and after diamondoid isolation. A GC-MS is used for the analysis step, where the isolated diamondoids are further studied to identify the type of diamondoid. The chromatogram shown in FIG. 3A represents the raw TIC of a crude oil sample before isolation. The chromatogram shown in FIG. 3B represents the TIC mass chromatograms of the crude oil sample after isolation, highlighting the series of adamantane peaks eluting first (marked by an arrow). This is further confirmed by the extracted mass spectrum shown in FIG. 3C, which verifies the purity of the isolated compounds and the effective removal of interferences from other cyclic hydrocarbons. The chromatogram shown in FIG. 3D represents the raw TIC of the rock extract samples before isolation of the diamondoids. The chromatogram shown in FIG. 3E represents the TIC mass chromatogram of the rock extract samples after isolation of the diamondoids. This highlights the series of diamantane peaks eluting second. This is further confirmed by the mass spectrum shown in FIG. 3F, which verifies the purity of the isolated compounds and the effective removal of interferences from other cyclic hydrocarbons.

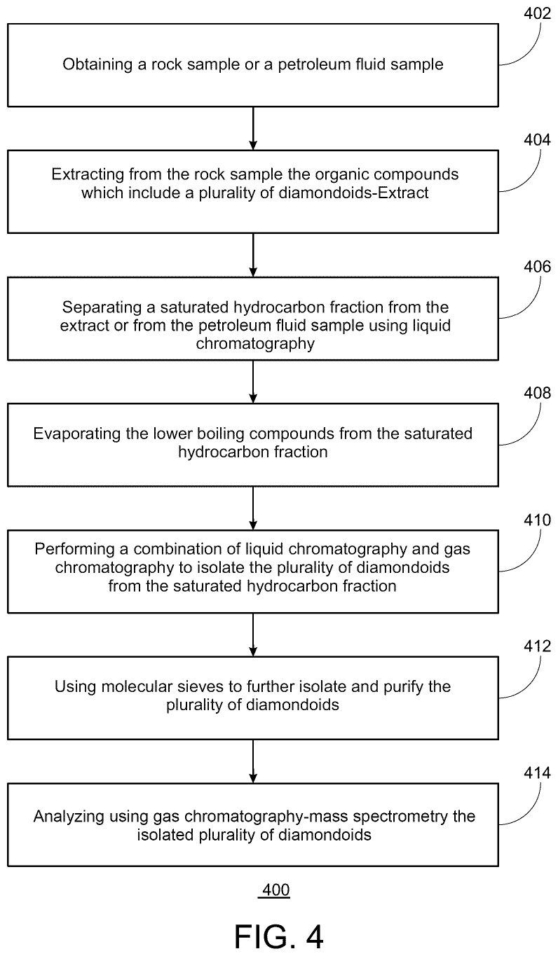

FIG. 4 is a process flow diagram representing the exemplary diamondoid isolation process.

At block 402, a rock sample or a petroleum fluid sample is obtained from a well rig, drill cuttings, soil samples, crude oil, or a refinery.

At block 404, a solvent extraction process is used to remove the organic compounds from the rock sample. In the case of a rock sample, the rock is crushed and on organic solvent is used to remove an extract that includes organic compounds and a plurality of diamondoids.

At block 406, a saturated hydrocarbon fraction is separated from the extract obtained from the rock sample of the previous step, using liquid chromatography separation technique. For petroleum fluid samples, LC is directly used to separate a saturated hydrocarbon fraction. A separate extraction process is not performed for petroleum fluid samples. During this process asphaltenes, resins, and aromatic compounds are removed. The saturated hydrocarbon fraction contains the plurality of diamondoids and a mixture of alkanes.

At block 408, the lower boiling compounds are evaporated from the saturated hydrocarbon fraction from the previous step. Selective evaporation is conducted in a rotary evaporator under vacuum to remove the lower boiling solvents present in the saturated hydrocarbon fraction.

At block 410, the saturated hydrocarbon fraction containing the plurality of diamondoids undergoes a combination of liquid chromatography and gas chromatography to isolate the plurality of diamondoids. The gas chromatography operates under a specific temperature regime with a constant carrier gas flow.

At block 412, the mixture of a plurality diamondoids and other hydrocarbons obtained at block 410 is flowed through a column packed with molecular sieves. In some implementations, zeolites are used as molecular sieves. This step further isolated the plurality of diamondoids and purifies it.

At block 414, the obtained plurality of diamondoids is analyzed using a gas chromatography-mass spectrometry to confirm the type of diamondoids isolated from the sample. This step also analyzes the vaporized solvents from the evaporation step at block 408, to confirm the absence of diamondoids.

An embodiment here provides a method that involves a three-step process for the extraction and purification of diamondoid compounds. These steps include preparation, isolation, and analysis of high purity diamondoids.

An implementation described here includes obtaining a rock sample or a petroleum fluid sample from a well rig or a refinery as a preparation step. The obtained rock sample undergoes a solvent extraction process to remove the organic compounds that include a plurality of diamondoids. Subsequently, a saturated hydrocarbon fraction is separated from the organic compounds obtained from the solvent extraction process as part of the preparation step. Petroleum fluid samples undergo an LC process directly for obtaining a saturated hydrocarbon fraction. Asphaltenes, resins, and aromatic compounds are removed from the saturated hydrocarbon fraction.

An implementation described here includes an isolation step. In this step the saturated hydrocarbon fraction undergoes selective evaporation to remove the lower boiling compounds. Following this, the saturated hydrocarbon fraction undergoes a combination of liquid chromatography and gas chromatography to isolate the plurality of diamondoids. In some implementations, the isolated plurality of diamondoids undergo a molecular sieving technique, to selectively capture the plurality of diamondoids. In implementations disclosed here, the obtained plurality of diamondoids is analyzed using a gas chromatography-mass spectrometry. This step helps to identify the type of diamondoids obtained from the various rock and petroleum fluid samples.

An embodiment described here provides a system for the isolation of a plurality of diamondoids from a rock sample or a petroleum fluid sample. The system includes an extraction vessel, in which the rock sample is placed. The extraction process includes the removal of organic compounds comprising a plurality of diamondoids to produce an extract. A first column is placed downstream of the extraction vessel which receives the extract from the extraction vessel. A saturated hydrocarbon fraction is removed from the extract by liquid chromatography (LC). For petroleum fluid samples, LC is directly used to remove the saturated hydrocarbon fraction. An evaporation column is placed downstream of the first column, which receives the saturated hydrocarbon fraction from the first column. A selective evaporation process removes a plurality of lower boiling point compounds to form a mixture of hydrocarbon fraction. A second column placed downstream of the first column receives the mixture of hydrocarbon fraction. The second column includes a high sorbent stationary phase. From the mixture of hydrocarbon fraction, the plurality of diamondoids is isolated by a combination of LC and gas chromatography (GC). A third column placed downstream of the second column includes a plurality of molecular sieves. The third column receives the plurality of diamondoids from the second column.

Other implementations are also within the scope of the following claims.

### EXEMPLARY EMBODIMENTS

The exemplary embodiments include:

1. A diamondoid isolation method comprising:
obtaining a rock sample or a petroleum fluid sample that comprises a plurality of diamondoids from a well rig or a refinery;
subjecting the rock sample or the petroleum fluid sample to programmed temperature and pressure cycles;
extracting a plurality of organic compounds from the rock sample using a first solvent to obtain an extract, wherein the extract comprises the plurality of diamondoids;
separating a saturated hydrocarbon fraction from the extract or the petroleum fluid sample using a second solvent by liquid chromatography (LC), wherein the saturated hydrocarbon fraction comprises a mixture of saturated hydrocarbons, the plurality of diamondoids, and the second solvent;
isolating the plurality of diamondoids from the saturated hydrocarbon fraction using a column packed with a high sorbent stationary phase, wherein the plurality of diamondoids is retained in the high sorbent stationary phase;
releasing the plurality of diamondoids from the high sorbent phase using a combination of gas chromatography (GC), LC, and molecular sieves; and
analyzing the plurality of diamondoids by a gas chromatography mass spectrometer (GC-MS) to identify the type of the plurality of diamondoids.

2. The method of embodiment 1, wherein the sample comprises rock core, drill cuttings, soil sample, downhole sampled petroleum fluids, produced crude oils, or kerogen samples.

3. The method of embodiment 1 or 2, wherein the first solvent comprises an organic solvent.

4. The method of embodiment 3, wherein the organic solvent comprises hexane, pentane, dichloromethane, isooctane, carbon disulfide, methanol, ethanol, chloroform, or mixtures thereof.

5. The method of any of embodiments 1 to 4, wherein the saturated hydrocarbon fraction comprises a mixture of normal alkanes and isoalkanes, cycloalkanes, and the plurality of diamondoids.

6. The method of any of embodiments 1 to 5, further comprising removing the second solvent and condensing the second solvent.

7. The method of embodiment 6, wherein analyzing the second solvent after condensing for the presence of the plurality of diamondoids, by a GC-MS.

8. The method of any of embodiments 1 to 7, wherein the plurality of diamondoids comprises adamantane, diamantane, triamantane, and alkylated variants thereof.

9. The method of any of embodiments 1 to 8, wherein the molecular sieves comprise a zeolite for selectively capturing the plurality of diamondoids.

10. The method of any of embodiments 1 to 9, further comprising:
immersing the zeolite in a third solvent, resulting in a mixture of zeolite and third solvent; and
sonicating and centrifuging the mixture of zeolite and third solvent to separate the plurality of diamondoids from the zeolite.

11. A diamondoid isolation device comprising:
a rock sample or a petroleum fluid sample;
an extraction vessel, wherein a rock sample undergoes an extraction process to remove organic compounds comprising a plurality of diamondoids, to form an extract;
a first column configured to receive the extract from the extraction vessel or the petroleum fluid sample, wherein a saturated hydrocarbon fraction is removed from the extract or the petroleum fluid sample by liquid chromatography (LC);
an evaporation column configured to receive the saturated hydrocarbon fraction from the first column, wherein by a selective evaporation process a plurality of lower boiling point compounds is removed from the saturated hydrocarbon fraction to form a mixture of hydrocarbon fraction;
a second column comprising a high-sorbent stationary phase, configured to receive the mixture of hydrocarbon fraction, wherein from the mixture of hydrocarbon fraction the plurality of diamondoids is isolated by a combination of LC and gas chromatography (GC); and
a third column comprising a plurality of activated molecular sieves, configured to receive the plurality of diamondoids from the second column.

12. The diamondoid isolation device of embodiment 11, wherein the plurality of diamondoids comprises adamantane, diamantane, triamantane, and alkylated variants thereof.

13. The diamondoid isolation device of embodiment 11 or 12, wherein the first column removes asphaltenes, polar compounds, and aromatic compounds from the extract by LC and leaves behind a mixture of normal and isoalkanes, cycloalkanes, and the plurality of diamondoids.

14. The diamondoid isolation device of any of embodiments 11 to 13, wherein the high-sorbent stationary phase comprises silica gel, nanotube materials, activated carbon, hyper crosslinked polymeric resins, or a combination thereof.

15. The diamondoid isolation device of any of embodiments 11 to 14, wherein the third column comprises a plurality of zeolites which selectively captures the plurality of the diamondoids.

16. The diamondoid isolation device of any of embodiments 11 to 15, wherein the plurality of diamondoids from the third column undergo further solvent extraction by LC, sonication, centrifugation, and evaporation to obtain the plurality of diamondoids with a high purity.

17. A diamondoid isolation method comprising:
obtaining a rock sample or a petroleum fluid sample from an oil well rig or a refinery;
extracting from the rock sample or the petroleum fluid sample organic compounds comprising a plurality of diamondoids using a solvent resulting in an extract;
isolating the plurality of diamondoids from the extract using a combination of liquid chromatography and gas chromatography;
purifying the plurality of diamondoids by molecular sieves; and
analyzing the type of the plurality of diamondoids by gas chromatography mass spectrometry.

18. The diamondoid isolation method of embodiment 17, wherein the plurality of diamondoids comprises adamantane, diamantane, triamantane, and alkylated variants thereof.

19. The diamondoid isolation method of embodiment 17 or 18, further comprising before isolating, selectively evaporating the extract to remove a plurality of lower boiling compounds.

20. The diamondoid isolation method of any of embodiments 17 to 19, wherein the molecular sieves comprise a plurality of zeolites.

What is claimed is:

1. A diamondoid isolation method comprising:
obtaining a rock sample or a petroleum fluid sample that comprises a plurality of diamondoids from a well rig or a refinery;
subjecting the rock sample or the petroleum fluid sample to programmed temperature and pressure cycles;
extracting a plurality of organic compounds from the rock sample using a first solvent to obtain an extract, wherein the extract comprises the plurality of diamondoids;
separating a saturated hydrocarbon fraction from the extract and the petroleum fluid sample using a second solvent by liquid chromatography (LC), wherein the saturated hydrocarbon fraction comprises a mixture of saturated hydrocarbons, the plurality of diamondoids, and the second solvent;
isolating the plurality of diamondoids from the saturated hydrocarbon fraction using a column packed with a high sorbent stationary phase, wherein the plurality of diamondoids is retained in the high sorbent stationary phase;
releasing the plurality of diamondoids from the high sorbent phase using a combination of gas chromatography (GC), LC, and molecular sieves; and
analyzing the plurality of diamondoids by a gas chromatography mass spectrometer (GC-MS) to identify the type of the plurality of diamondoids.

2. The method of claim 1, wherein the sample comprises rock core, drill cuttings, soil sample, downhole sampled petroleum fluids, produced crude oils, or kerogen samples.

3. The method of claim 1, wherein the first solvent comprises an organic solvent.

4. The method of claim 3, wherein the organic solvent comprises hexane, pentane, dichloromethane, isooctane, carbon disulfide, methanol, ethanol, chloroform, or mixtures thereof.

5. The method of claim 1, wherein the saturated hydrocarbon fraction comprises a mixture of normal alkanes and isoalkanes, cycloalkanes, and the plurality of diamondoids.

6. The method of claim 1, further comprising removing the second solvent and condensing the second solvent.

7. The method of claim 6, wherein analyzing the second solvent after condensing for the presence of the plurality of diamondoids, by a GC-MS.

8. The method of claim 1, wherein the plurality of diamondoids comprises adamantane, diamantane, triamantane, and alkylated variants thereof.

9. The method of claim 1, wherein the molecular sieves comprise a zeolite for selectively capturing the plurality of diamondoids.

10. The method of claim 9, further comprising:
immersing the zeolite in a third solvent, resulting in a mixture of zeolite and third solvent; and
sonicating and centrifuging the mixture of zeolite and third solvent to separate the plurality of diamondoids from the zeolite.

11. A diamondoid isolation device comprising:
a rock sample or a petroleum fluid sample;
an extraction vessel, wherein the rock sample undergoes an extraction process to remove organic compounds comprising a plurality of diamondoids, to form an extract;
a first column configured to receive the extract from the extraction vessel or the petroleum fluid sample wherein a saturated hydrocarbon fraction is removed from the extract or the petroleum fluid sample by liquid chromatography (LC);
an evaporation column configured to receive the saturated hydrocarbon fraction from the first column, wherein by a selective evaporation process a plurality of lower boiling point compounds is removed from the saturated hydrocarbon fraction to form a mixture of hydrocarbon fraction;
a second column comprising a high-sorbent stationary phase, configured to receive the mixture of hydrocarbon fraction, wherein from the mixture of hydrocarbon fraction the plurality of diamondoids is isolated by a combination of LC and gas chromatography (GC); and
a third column comprising a plurality of activated molecular sieves, configured to receive the plurality of diamondoids from the second column.

12. The diamondoid isolation device of claim 11, wherein the plurality of diamondoids comprises adamantane, diamantane, triamantane, and alkylated variants thereof.

13. The diamondoid isolation device of claim 11, wherein the first column removes asphaltenes, polar compounds, and aromatic compounds from the extract by LC and leaves behind a mixture of normal and isoalkanes, cycloalkanes, and the plurality of diamondoids.

14. The diamondoid isolation device of claim 11, wherein the high-sorbent stationary phase comprises silica gel, nanotube materials, activated carbon, hyper crosslinked polymeric resins, or a combination thereof.

15. The diamondoid isolation device of claim 11, wherein the third column comprises a plurality of zeolites which selectively captures the plurality of the diamondoids.

16. The diamondoid isolation device of claim 15, wherein the plurality of diamondoids from the third column undergo further solvent extraction by LC, sonication, centrifugation, and evaporation to obtain the plurality of diamondoids with a high purity.

17. A diamondoid isolation method comprising:
obtaining a rock sample or a petroleum fluid sample from an oil well rig or a refinery;
extracting from the rock sample or the petroleum fluid sample organic compounds comprising a plurality of diamondoids using a solvent resulting in an extract;
isolating the plurality of diamondoids from the extract using a combination of liquid chromatography and gas chromatography;
purifying the plurality of diamondoids by molecular sieves; and
analyzing the type of the plurality of diamondoids by gas chromatography mass spectrometry.

18. The diamondoid isolation method of claim 17, wherein the plurality of diamondoids comprises adamantane, diamantane, triamantane, and alkylated variants thereof.

19. The diamondoid isolation method of claim 17, further comprising before isolating, selectively evaporating the extract to remove a plurality of lower boiling compounds.

20. The diamondoid isolation method of claim 17, wherein the molecular sieves comprise a plurality of zeolites.
